(19) United States
(12) Patent Application Publication
(10) Pub. No.: US 2004/0019366 A1
(43) Pub. Date: Jan. 29, 2004

(54) System and method for classification of cardiac activity using multiple sensing locations

(76) Inventors: William B. Rottenberg, Durango, CO (US); Scott Mathis, Durango, CO (US)

Correspondence Address: Gottlieb, Rackman & Reisman, P.C., 8th Floor, 270 Madison Avenue, New York, NY, 10016-0601, US

(21) Appl. No.: 10/205,836

(22) Filed: Jul. 25, 2002

(51) Int. Cl. A61N 1/362

(52) U.S. Cl. 607/25

(57) ABSTRACT

In a cardiac system with a multi-electrode lead, the intrinsic activity at each electrode is sensed and used to generate a wavefront profile. The timing, relative and absolute duration of events within the profile and the location of the first electrode at which an event is sensed during a cardiac cycle are used to generate a signal indicative of the cardiac condition of the patient.

### RELATED APPLICATIONS

The subject application is related to the subject matter disclosed in the following applications and incorporated herein by reference:

Cardiac Electrode Catheter and Method of Manufacturing the Same, Ser. No. 09/761,333, filed Jan. 1, 2001, now ______;

Improved Leads for the Treatment of Patients with CHF, Ser. No. 10/135,161, filed, Apr. 29, 2002, now ______;

Method and Apparatus for Manufacturing Implantable Electrodes Having Controlled Surface and Integral Conductor, Ser. No. ______, filed May 24, 2002, now ______.

Cardiac Stimulation Apparatus and Method for Treatment of Atrial Fibrillation, Ser. No. 10/134,197, filed Apr. 26, 2002, now ______;

Method and Apparatus for Determining Spatial Relation of Multiple Implantable Electrodes, Ser. No. 10/132,862, filed Apr. 25, 2002, now ______.

### BACKGROUND OF THE INVENTION

A. Field of Invention

This invention pertains to a method and apparatus for classifying the cardiac activity of a patient using an implantable multi-electrode lead. More particularly, the invention pertains to an implantable cardiac device in which the timing of intrinsic electrical signals sensed by various electrodes is used as a criteria for determining when therapy is indicated.

B. Description of the Prior Art

Implantable cardiac devices, such as pacemakers, are essentially pulse generators that must include electronic circuitry for determining automatically when therapeutic electric pulses are required. Typical cardiac devices utilize a lead with two electrodes located in a single chamber, the electrodes being spaced at relatively short distance from each other. The two electrodes are used to sense intrinsic cardiac activity, with one of the electrodes being a reference electrode. Thus, this arrangement is only capable of sensing cardiac activity only at one location within the heart.

Dual chamber devices are also known which utilize one or two leads with two electrodes disposed in each cardiac chamber. Again, the electrodes in each chamber are spaced at a relatively short distance to each other. Therefor the only information that can be obtained through these electrodes is the time of occurrence of an event. Intrinsic activity or events on these electrodes are measured by these devices on then used in complex algorithms to detect the cardiac condition of the patient. This in turns adds an additional level of complexity to the devices. In addition, there is also a degree of uncertainty associated with the algorithm and hence, the clinicians may not have full confidence in the ability of these devices to operate properly.

The following references provide descriptions and discussions of the complex algorithms presently in use.

U.S. Pat. Nos. 6,212,428 and 6,275,732 describe a multi-stage morphology-based ventricular tachycardia detection system. The multi-stage approach is based on a computational complexity as the priority basis for the stages. Easier algorithms are implemented first, with more complex algorithms added if further identification is required. The patents mention the use of the R wave width as one of the sensed parameters, but do not mention any specific method for obtaining this parameter. With single-electrode systems, a type of far-field signal can be collected using the atrial and ventricular electrodes and the far-field R wave width could be measured with this technique. Neither patent addresses how to handle of natural R wave variations associated with certain conditions, such as exercise. They also mention a multi-electrode lead, but do not describe how many electrodes, where the electrodes are, or how they may be used for sensing.

U.S. Pat. No. 4,354,497 describes a multi-electrode sensing system for identification of ectopic focus location. It describes a system that essentially senses from two ventricular sites; the inter-ventricular septum and a combination of sensing electrodes spaced elsewhere in the heart. The outputs of the other electrodes are coupled so that the logic circuitry cannot determine whether an event is sensed from any one particular electrode. The patent also states that a normal R wave depolarization wavefront will be seen on the IV septum before anywhere else in the ventricle, so it looks for the sequence of events between the one IV electrode and the remaining others. This is very simplistic approach with many disadvantages. For example, a major shortcoming is that ectopic events that occur near the IV electrode could be identified as intrinsic.

U.S. Pat. No. 6,078,837 describes a system for atrial fibrillation treatment that uses multiple sensing and pacing electrodes. There is no discussion of detection methods using the multiple sensing electrodes. They are used to determine pulse timing in relation to the fibrillation wavefront.

In the article by Walsh, Singer, Mercando & Furman entitled ‘Differentiation of Arrhythmias in the Dog by Measurement of Activation Sequence Using an Atrial and Two Ventricular Electrodes’ PACE November 1988, Part II, Vol. 11, 1732-1738, a method is disclosed using three epicardial electrodes: one left atrial, one right ventricular and, one left ventricular. Two intervals, LA-LV and LV-RV, are measured and the intervals are used to classify events as ectopic or intrinsic in origin. This article uses impractical electrode locations, and does not address or even discuss variable problems such as exercise, and does not attempt to identify the actual location of the ectopic origin.

Template matching is also known using a single electrode and digitization of the wavefront at that electrode. Information provided by far-field sensing at that location provides some measure of the type of event, ectopic or intrinsic. No information about the location of the origin can be extracted from a single point.

Various other known sensing methods offer different degrees of specificity and success, with varying amounts of computational requirements, including digitization of the signal which is, in itself, power intensive. Moreover, exercise tolerance is not covered by any of these methods. See Steinhouse, Wells, Greenhut, Maas, Nappholz, Jenkins & Dicarlo ‘Detection of Ventricular Tachycardia Using Scanning Correlation analysis’, PACE, December 1990, Part II, Vol. 13. 1930-1936; Langberg, Gibb, Auslander & Griffin ‘Identification of ventricular tachycardia with use of the morphology of the endocardial electrogram’ Circulation 1988. 77, No. 6, 1363-1369; Lin, Dicarlo, & Jenkins ‘Identification of Ventricular Tachycardia Using Intracavitary Ventricular Electrograms: Analysis of Time and Frequency Domain Patterns’ PACE, November 1988, Part I, Vol 11, 1592-1606; Saba, Gorodeski, Yang, MacAdam, Link, Homoud, Estes III, & Wang ‘Use of Correlation Waveform Analysis in Discrimination Between Antegrade and Retrograde Atrial Electrogram During Ventricular Tachycardia’ J Cardiovasc Electrophysiol. Vol. 12, 145-149, February 2001.

### SUMMARY OF THE INVENTION

Briefly, the present invention pertains to a cardiac system comprising a pulse generator and a multi-electrode assembly, and sensor circuitry adapted to detect signals through the electrodes and to generate a cardiac classification. This classification is then used for determining what therapy, if any, is to be applied to the heart.

The improved capabilities of sensing available with multi-site sensing allow more accurate and quicker determinations of a tachyarrhythmia. Current systems that have to analyze rate or chamber synchrony characteristics can require several seconds to identify a tachyarrhythmia. During this time, the patient can easily become symptomatic because an inappropriate therapy may be being delivered while the diagnosis is being made. With multi-site sensing, the determination can be made on the first cycle, allowing proper therapy to be initiated much more quickly.

### BRIEF DESCRIPTION OF THE DRAWINGS

FIG. 1 shows a cardiac device implanted in a patient and constructed in accordance with this invention;

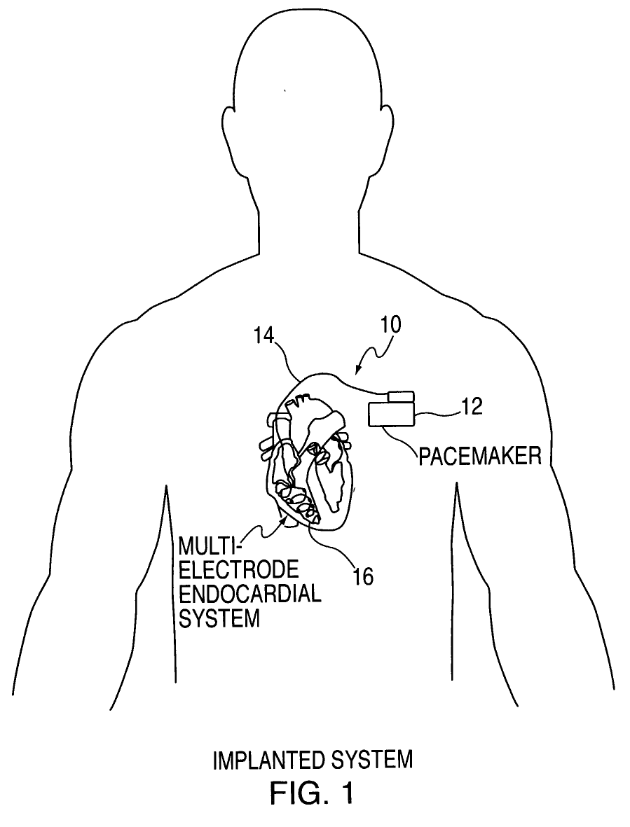

FIG. 2 shows an enlarged isometric view of the multi-electrode lead used in the device of FIG. 1;

FIG. 3 shows a partial sectional view of a heart with a first multi-electrode lead;

FIG. 4 shows a partial sectional view of a heart with a second multi-electrode lead;

FIG. 5 shows a block diagram of a sensing circuit constructed in accordance with this invention;

FIG. 6 shows details of the block diagram of FIG. 5;

FIG. 7 shows a block diagram of another embodiment for the sensing circuit;

FIG. 8 shows a somewhat diagrammatic sectional view of a patient's heart with a multi-electrode lead in the right ventricle;

FIG. 9 shows a wavefront characteristic of a sinus ventricular event as sensed through the multi-electrode lead of FIG. 8;

FIG. 10 shows a wavefront characteristic of a PVC event as sensed through the multi-electrode lead of FIG. 9;

FIG. 11 shows a somewhat diagrammatic sectional view of a patient's heart with the locations of the electrodes in the right atrium;

FIG. 12 shows a wavefront typical of a sinus atrial event as sensed through the multi-electrode lead of FIG. 11;

FIG. 13 shows a wavefront typical of a PAC event as sensed through the multi-electrode lead of FIG. 11;

FIG. 14 shows wavefront typical of a retrograde P event as sensed through the multi-electrode lead of FIG. 11;

FIG. 15 shows a flow chart for the operation of a device with a multi-electrode lead in accordance with this invention; and

FIG. 16 shows a flow chart with details of the analysis performed on the array obtained in FIG. 15.

### DETAILED DESCRIPTION OF THE INVENTION

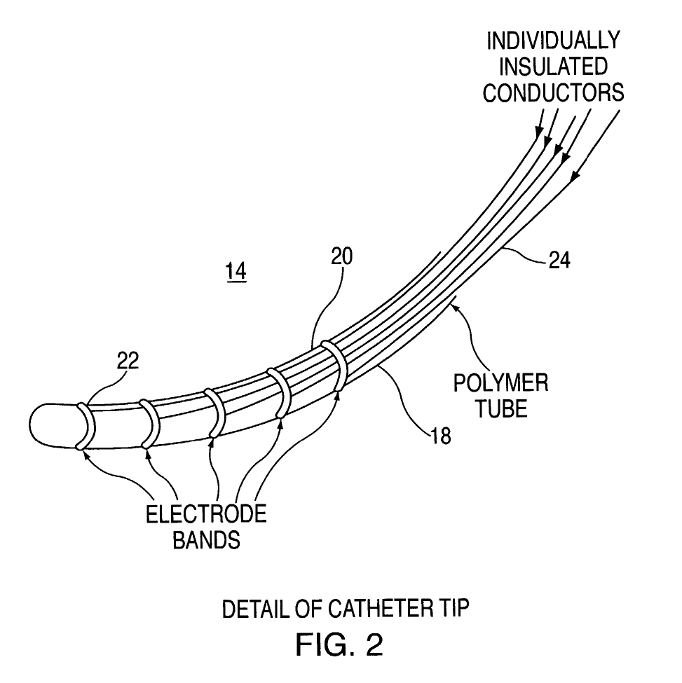

Referring now to the Figures, an implantable cardiac device 10 includes a pacemaker 12 and a multi-electrode lead 14. The multi-electrode lead has a proximal end connected to the pacemaker 12 and a distal end extending into the atrium and the ventricle of a patient's heart 16. As shown in more detail in FIG. 2, the lead 14 consists of an elongated member or tube 18 which is provided at its distal end 20 with a plurality of electrodes 22 preferably disposed circumferentially about the tube 18. Each electrode consists of a wire 24 that is wrapped around the tube 18 and then extends through a hole in the tube into the interior space contained therein and extend to the proximal end of the tube (not shown). Details of manufacturing this multi-electrode lead are described in the above identified patent application Ser. No. 09/761,333.

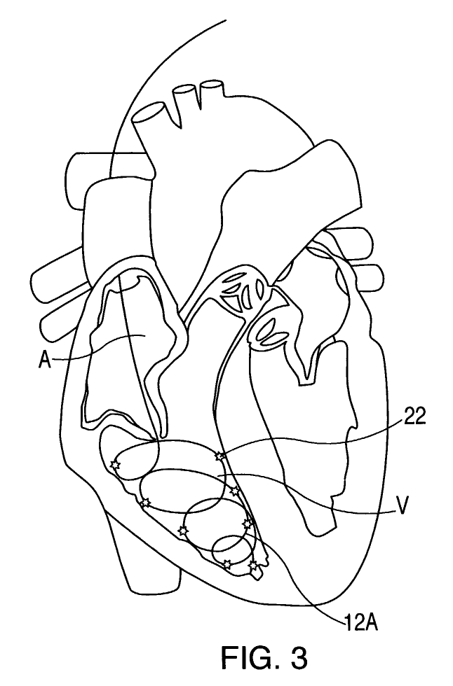

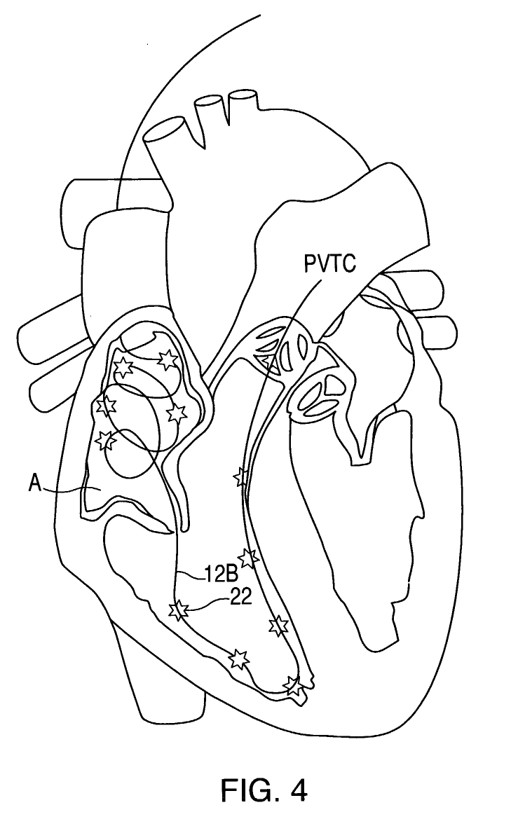

The lead 12 can be preformed so that when it is implanted, it takes a certain shape selected to position the electrodes in contact with various preselected tissues of the heart. For example, in FIG. 3, a lead 12A is shown that has a generally spiral shape with the electrodes contacting the walls of the ventricle V. In FIG. 4 the lead 12B has two portions: a first portion having electrodes and disposed in a spiral shape in the atrium A and a second portion having V-shape so that it extends through the tricuspid valve TV to the apex of the ventricle, along the free wall and then along the septal wall to the RVOT. A more detailed description of the lead and how it is positioned in the cardiac chambers for providing CHF therapy is found in the above-mentioned application Ser. No. 10/135,161.

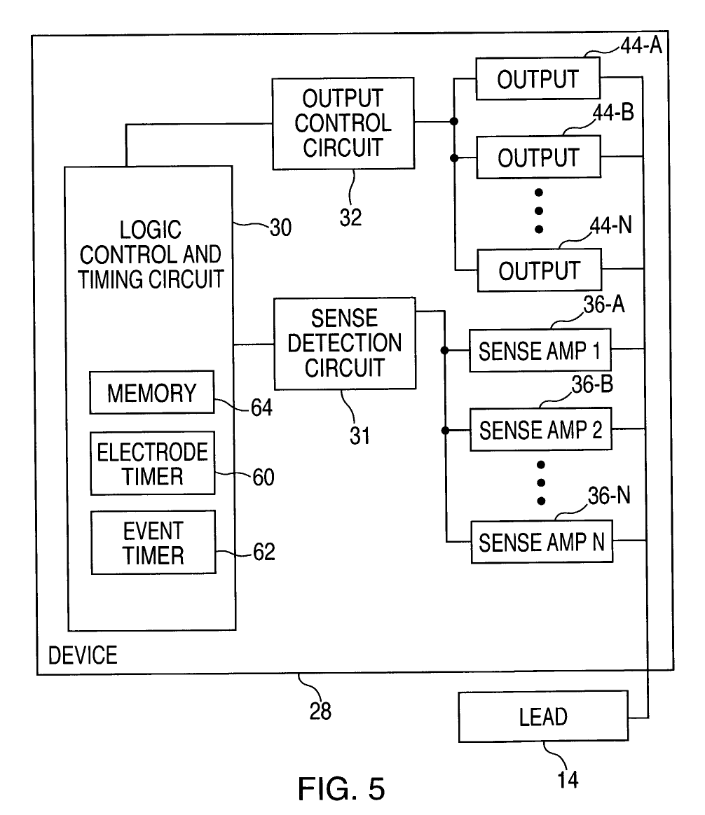

The present invention pertains to a novel technique for sensing intrinsic cardiac signals in the heart. For this purpose, the pacemaker is provided with a sense circuit shown in the block diagram of FIG. 5. The sense circuit 28 includes a logic control and timing diagram circuit 30, a sense detection circuit 31 and a plurality of sense amplifiers 36-A, 36-B . . . 36-N where N is the number of designated sense electrodes. These amplifiers are then connected to the respective sense electrodes of lead 14. Hence, in this circuit 28 each amplifier 36 is associated with a single sense electrode. The amplifiers amplify and filter these signals. The common sense detection pacemaker 12 and a distal end extending into the atrium and the ventricle of a patient's heart 16. As shown in more detail in FIG. 2, the lead 14 consists of an elongated member or tube 18 which is provided at its distal end 20 with a plurality of electrodes 22 preferably disposed circumferentially about the tube 18. Each electrode consists of a wire 24 that is wrapped around the tube 18 and then extends through a hole in the tube into the interior space contained therein and extend to the proximal end of the tube (not shown). Details of manufacturing this multi-electrode lead are described in the above identified patent application Ser. No. 09/761,333.

The lead 12 can be preformed so that when it is implanted, it takes a certain shape selected to position the electrodes in contact with various preselected tissues of the heart. For example, in FIG. 3, a lead 12A is shown that has a generally spiral shape with the electrodes contacting the walls of the ventricle V. In FIG. 4 the lead 12B has two portions: a first portion having electrodes and disposed in a spiral shape in the atrium A and a second portion having V-shape so that it extends through the tricuspid valve TV to the apex of the ventricle, along the free wall and then along the septal wall to the RVOT. A more detailed description of the lead and how it is positioned in the cardiac chambers for providing CHF therapy is found in the above-mentioned application Ser. No. ______ case 18.

The present invention pertains to a novel technique for sensing intrinsic cardiac signals in the heart. For this purpose, the pacemaker is provided with a sense circuit shown in the block diagram of FIG. 5. The sense circuit 28 includes a logic control and timing diagram circuit 30, a sense detection circuit 31 and a plurality of sense amplifiers 36-A, 36-B . . . 36-N where N is the number of designated sense electrodes. These amplifiers are then connected to the respective sense electrodes of lead 14. Hence, in this circuit 28 each amplifier 36 is associated with a single sense electrode. The amplifiers amplify and filter these signals. The common sense detection circuit 31 is provided to detect and analyze intrinsic cardiac signals from each electrode. The circuit 31 can be adapted to detect the duration of each intrinsic signal or event. The information thus generated is fed to the logic control and timing circuit 30. The circuit 30 then uses this information and a predetermined algorithm to determine what therapy, if any, is required. If therapy is required, the circuit 30 activates output controller 32 which then causes output pulses to be applied to the pacing electrodes of the lead 14 in a certain sequence through output circuits 44-A, 44-B etc.

The logic and timing circuit includes several components, such as an electrode timer 60, an event timer 62 and a memory 64. The purpose and operation of these elements is disclosed in more detail below.

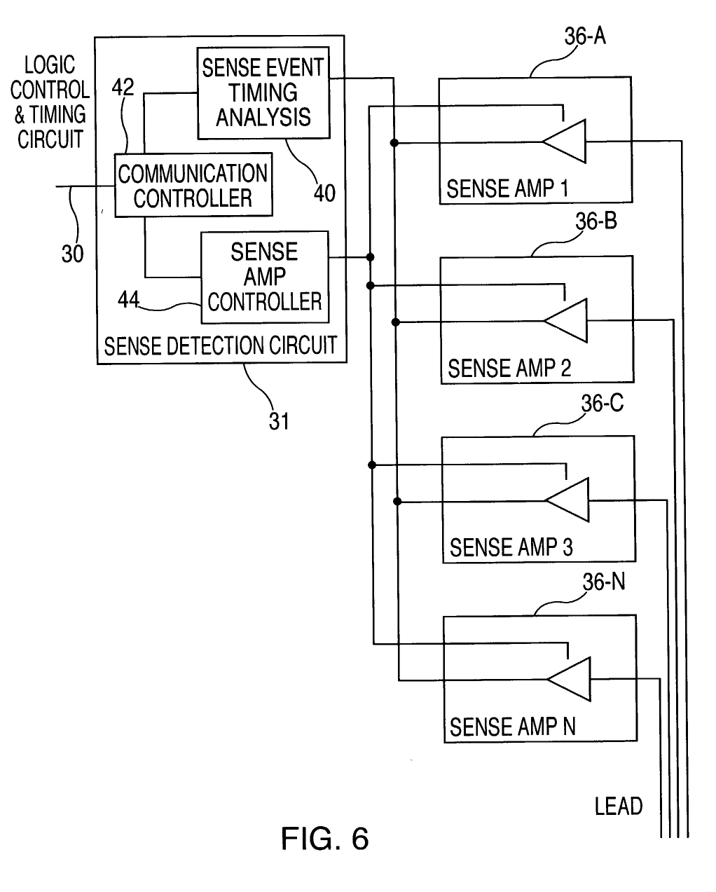

FIG. 6 shows more details of the embodiment of FIG. 5. As shown in this Figure, amplifiers 36-A, 36-B . . . 0.36-N, are independently controlled by the sense detection circuit 31 and have their own variable gain and filtering characteristics. The common sense detection circuit 32 sets the gains and filtering characteristics of each amplifier through a sense amplifier controller 44. A sense event timing analysis circuit 40 receives the sense event information from the amplifiers and compiles this information to indicate a moving wavefront, as discussed below. The communication controller 42 transmits this information to the logic control and timing circuit 30 (FIG. 5) and receives therefrom the parameters for the amplifiers.

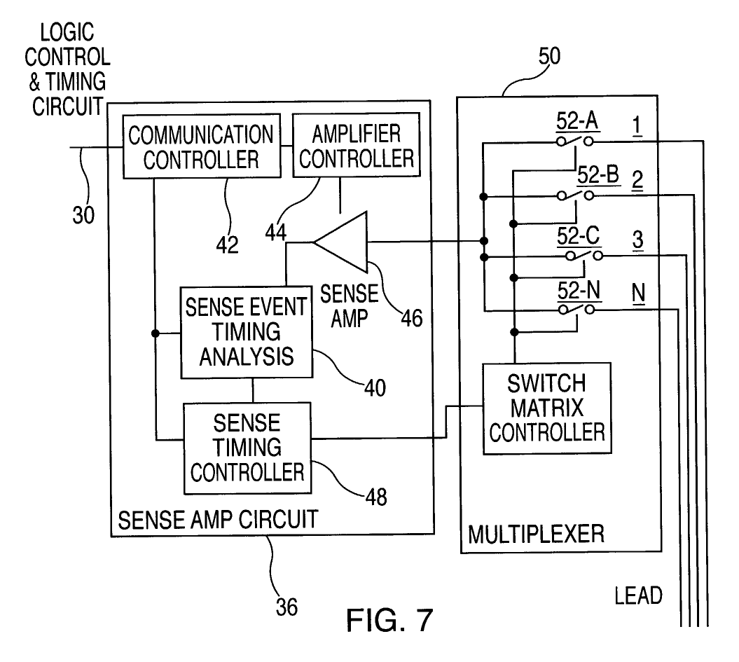

FIG. 7 shows another embodiment of the invention. In this embodiment, a sense amplifier circuit 36′ is provided that includes a single amplifier 46 as well as the communication controller 42, amplifier controller 44, sense event timing analyses circuit 40 and a sense timing controller 48. In addition, a multiplexer 50 is provided which includes a plurality of electronic control switches 52-A, 52-B. 52-C 52-N arranged in a matrix, which electrodes are controlled by a switch matrix controller 54 in accordance with control signals from the sense timing controller 48. Each switch is connected to a respective sensing electrode of lead 14. The switch matrix controller closes and opens each switch in a predetermined sequence to define windows during which the electrodes are monitored for intrinsic cardiac signals. The signals are amplified by the amplifier 46, filtered, analyzed by the sense event timing analysis circuit 40 and the resulting information is sent by the communication controller 42 to the logic control and timing circuit 30. Output signals are transmitted through the same or a different set of switches. Alternatively, the output signals are transmitted through individual amplifiers.

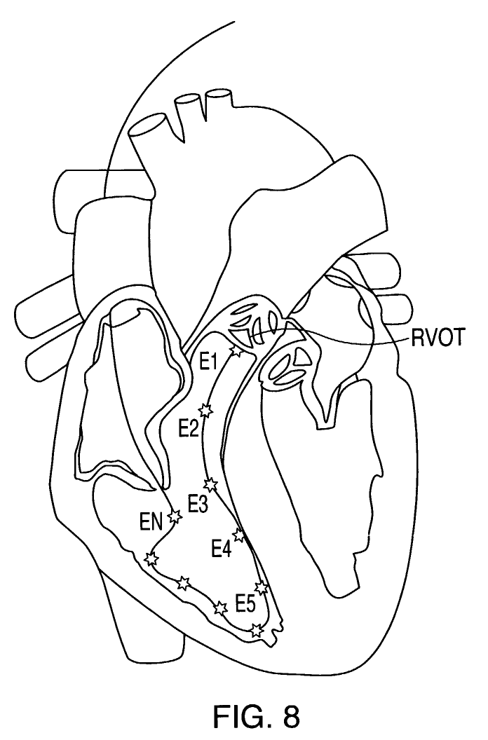

FIG. 8 shows a multi-electrode lead having a plurality of electrodes E1, E2, E3 . . . EN. In this embodiment, the most distal electrode, E1, is disposed in, or near the RVOT and the lead passes adjacent to the septal wall with the remaining electrodes are dispersed in the right ventricle.

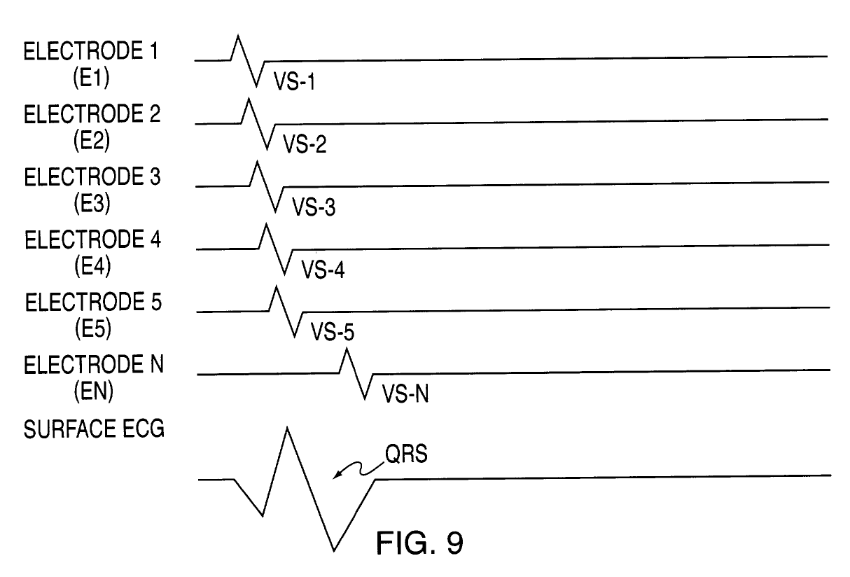

FIG. 9 shows the signals VR1-VRN sensed through the electrodes E1-EN of FIG. 8 during a sinus ventricular contraction occurs. The corresponding surface ECG of the respective QRS complex is shown for comparison. The signals VR1-VRN all have the same general shape, however, importantly, illustrate how a wavefront traveling through the cardiac tissues are sensed by the electrodes E1-EN. At least some of the electrodes may also pick up some far-field signals which are filtered or otherwise eliminated by the sensing circuits shown in FIGS. 5-7, and hence are not shown. The signals sensed at each electrode represent the sums of the monophasic action potentials near the respective electrodes. Details of how these wavefronts are analyzed are discussed in detail below.

Typically, the contraction starts high in the ventricle, near the RVTC and then propagates downward to the septum and around the free wall of the ventricle. The important feature of the wavefronts for the purposes of this invention, is that they are delayed in time. More specifically, the further an electrode is from the electrode closest to the site of the initial depolarization, the more delayed is the wavefront. This phenomenon occurs because it takes a finite time for the wavefront to propagate through the respective cardiac tissues. In the present invention, the occurrence and timing of the wavefronts are used to derive an indication of the current classification of the heart rate (i.e., tachycardia, fibrillation, bardycardia, etc.) as discussed in more detail below.

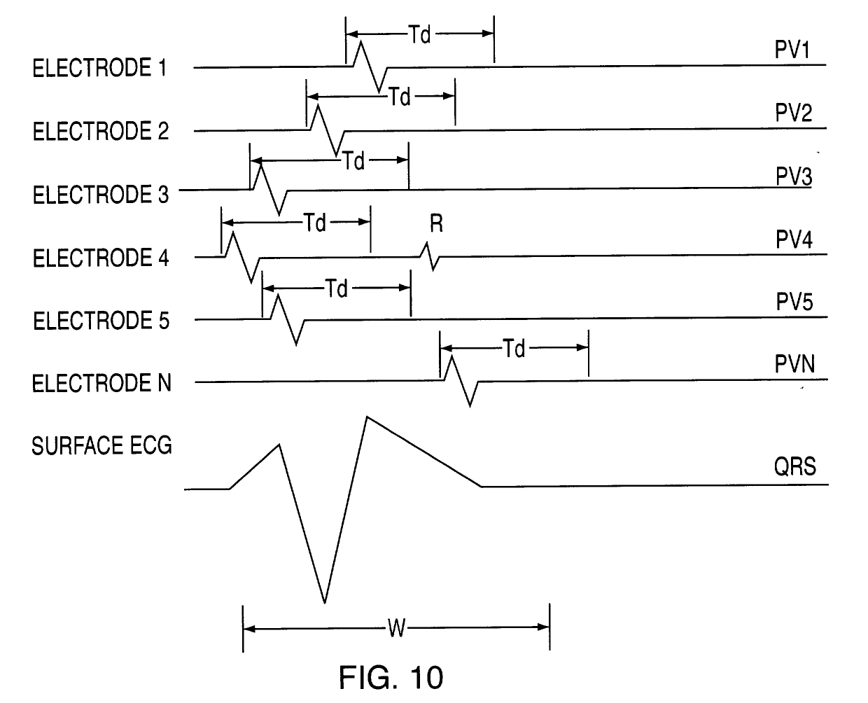

FIG. 10 illustrates the signals PV1-PVN sensed in electrodes E1-EN during a typical premature ventricular contraction (PVC). As can be seen in this figure, this type of contraction originated further down, near the septal wall, and therefore it is first sensed by electrode E4. The contraction then propagates through the cardiac tissues until it is sensed by the respective electrodes.

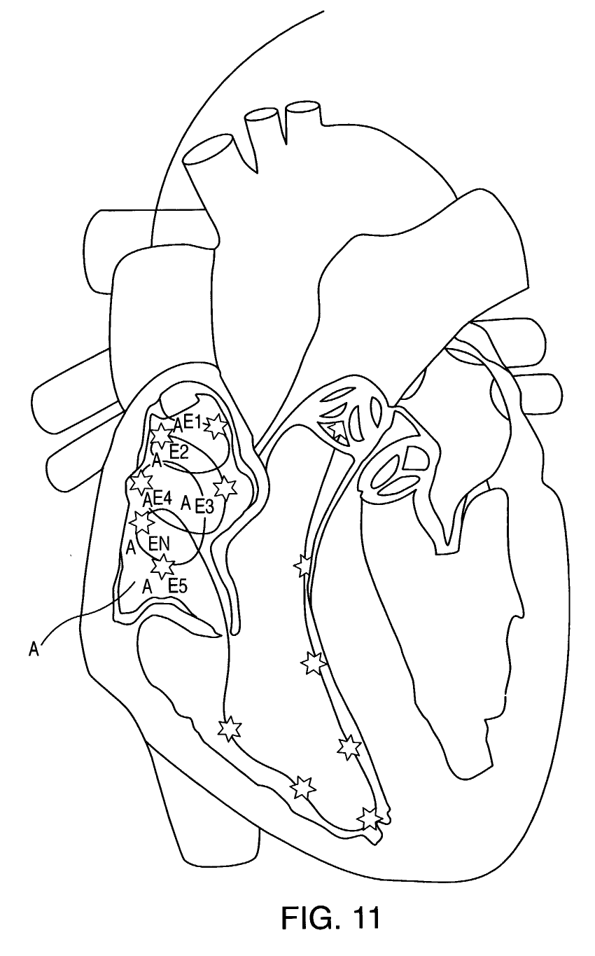

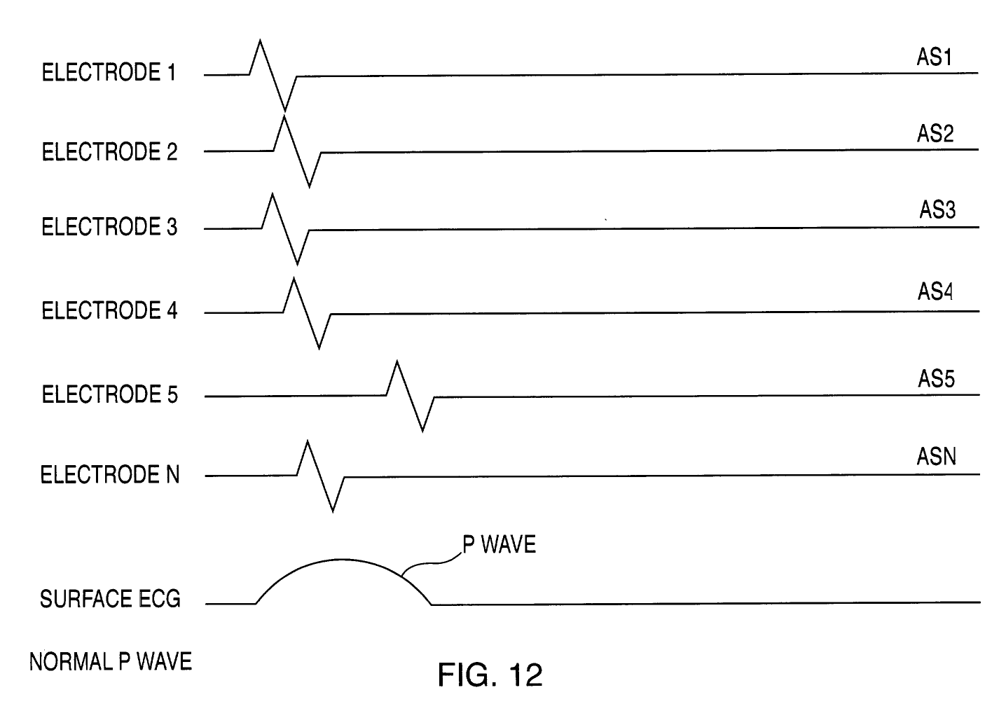

As discussed above, the multi-electrode lead may be provided with a portion disposed in the atrium. As shown more clearly in FIG. 11 the lead portion 12C is disposed in the atrium and has a spiral shape selected to insure that as many of the electrodes AE1, AE2, AE3, AE4, AE5, AEN are disposed in the atrium. FIG. 12 shows the signals AS1-ASN sensed in the respective atrial electrodes. The surface ECG—in this case a sinus P-wave—is shown as well for the sake of comparison. Typically, a sinus atrial contraction starts near the SA node, high in the atrium, and is first sensed by electrode AE1. It propagates along a fast conduction path to the AV node, located in the lower part of the atrium near the septum wall, and then at a slower rate to the rest of the atrium. This effect is clearly seen in FIG. 12.

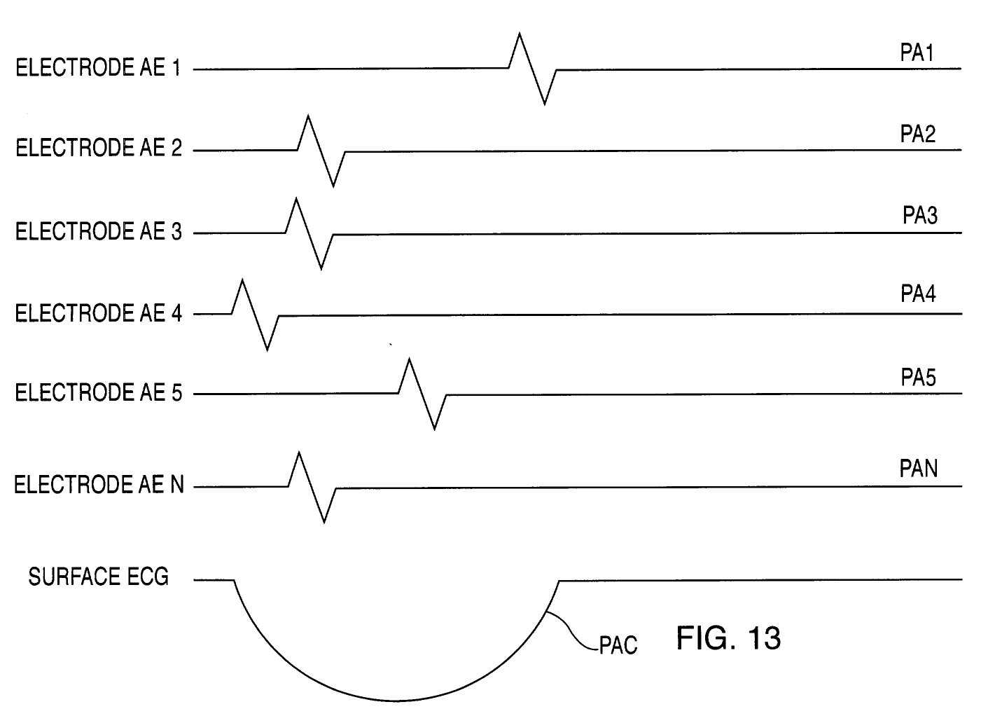

FIG. 13 shows the signals PA1-PAN corresponding to a preliminary atrial contraction (PAC) originating near electrode AE4 and propagates in a special pattern, as shown.

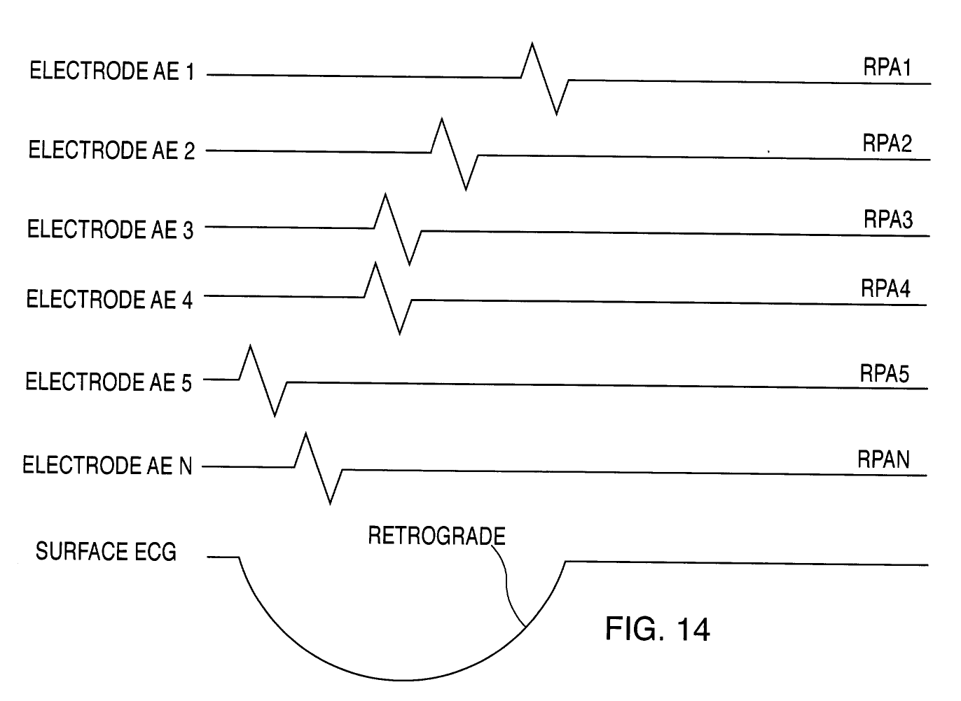

FIG. 14 shows a retrograde P wave due to a ventricular event. It originates near electrode AE5 and it propagates through the rest of the atrium A, with the electrodes AE1-AEN sensing respective signals RPA1-RPN. Note that the pattern of propagation for a retrograde P wave is different and at a slower rate then a sinus P wave.

Thus, as illustrated in the Figures, cardiac events in the cardiac chambers are sensed through the respective electrodes as signals. Based on where the first signal appears, the relative and absolute timing of these signals and the duration or repetition rate of the signals, patterns are identifiable, with each pattern being characteristic of a particular type of intrinsic cardiac activity.

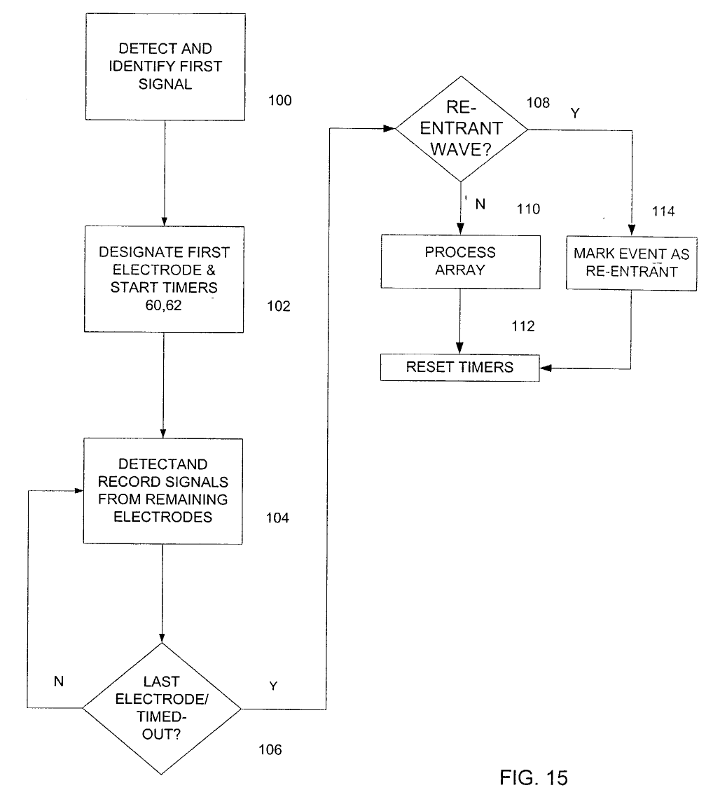

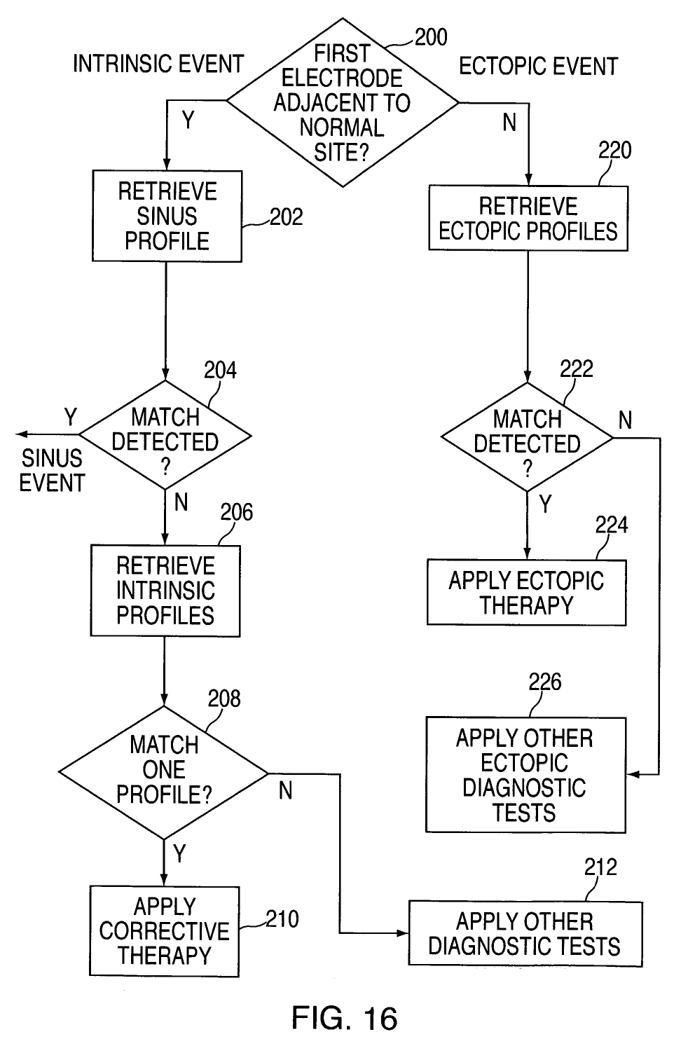

One scheme for analyzing the wavefronts is now described in conjunction with the block diagram of FIG. 5, and the flow charts of FIGS. 15 and 16, using the logic and control circuit 30 and the other circuits shown therein. More specifically, the sense amplifiers 36-A to 36-N are used to sense the signals on the respective electrodes of lead 14. These signals are then filtered and conditioned by the sense detection circuit to generate electrode signals that look like the idealized signals of FIGS. 8-14, or digital equivalents thereof. (In the following description, the elements of the logic control and timing circuit 30 are disclosed as being discrete analog elements for the sake of clarity, it being understood that the circuit 30 is preferably implemented digitally, using a microprocessor). The electrode timer 60 is used to define a sensing window W during which a wavefront occurs. The window W has fixed length that is either preprogrammed using a default value, or is programmed by the clinician during the setup phase of the device 12. The event timer 62 is used to measure the duration of an event.

The flow chart of FIG. 15 shows an overview of the operation of the device. For the purposes of this description it is assumed that a previous cycle has terminated and that both timers 60 and 62 has been reset. In step 100 a signal is detected on any one of the electrodes. Since timers 60 and 62 are reset, this signal is identified or designated as a first signal of a wavefront. In step 102 the respective electrode is designated as the first electrode and the timers 60 and 62 are started. In step 104 the signals from the other electrodes are detected and the data descriptive of the resulting wavefront is recorded in the form of an array. Different types of cardiac activities result in different arrays.

In step 106, a test is performed to determine if a signal has been received from all the electrodes. If not, then the collection and recondition of data continues in step 104. In order to insure that the device does not hang up in step 106 if a signal from an electrode is missed, or is not detected, timer 60 can be set so that it times out and resets itself after a predetermined time W, for example 200 ms, shown in FIG. 10. Then in step 106, instead of, or in addition to, checking for a last electrode, the electrode timer 60 is checked. If it has timed out (i.e., at the end of period W), it is assumed that all the signals have been received.

Alternatively, a separate timer 62 may be used for each electrode. Only one timer is active at any given time. Referring to FIG. 10, the timer for electrode e4 first starts since in this case electrode e4 is the initial electrode. Next, timer for electrode 3 is started and the timer for electrode e4 is turned off. Each timer is on for a predetermined time Td (e.g., 30 msec). The QRS complex is finished when the last timer (in this case, that of electrode N) times out.

Next, in step 108 a re-entrant wave, indicative of fibrillation, is recognized. One method of making this determination is to monitor during period W or Td the number of QRS complexes received. Under sinus condition a single QRS signal is present. Therefore, as the QRS complex is being logged (i.e., during W or Td in FIG. 10) the number of signals is counted. If more than one signals is detected then a re-entrant event is declared.

An alternative scheme to detect re-entrant waves is simply to monitor the respective timer 62 for each electrode. A signal sensed on an electrode that has its timer 62 running can be declared a re-entrant event. Of course, other methods for detecting re-entrant events may be used as well.

Getting back to FIG. 15, in step 108 the electrodes are being monitored for re-entrant events. Preferably while this process is occurring in step 110 the array or wavefront is analyzed. For example, if four events (from four electrodes) are received and a template or profile is recognized then the analysis is completed even before signals from all the electrodes are received. In this manner, the process does not have wait the signals from all the electrodes are received, because otherwise the system may be too slow to respond.

FIG. 16 shows more details of how the collected data is processed. For the purposes of this discussion, all events that start near the normal focus are considered intrinsic events and all other events are considered ectopic events. Starting with step 200, a check is made to determine if the first electrode designated in step 102 is close to a normal focus for contraction (i.e., the SA node in the atrium or the AV node in the ventricle). If it is then an intrinsic event is assumed.

The memory 64 is used to store several profiles of different types of events. These profiles may be provided by the manufacturer, may be programmed in by the cardiologist, or may be derived dynamically using the past history of the patient.

Getting back to FIG. 18, in step 202 a profile characteristic of sinus cardiac activity is retrieved from memory 64 and in step 204 this profile is compared to the array generated from the latest wavefront. If a match is detected then a sinus cardiac condition is declared and no therapy is provided. As discussed above, a match may be detected even before the signals from all the electrodes are collected. If a match is not detected, then in step 206 profiles characteristic of other intrinsic events are retrieved from the memory 64. In step 208, the profiles are compared to the array from the latest wavefront. If a match is found with a particular profile then the current cardiac condition of the patient has been identified and in step 210 the appropriate corrective therapy is applied. If no match is found in step 208, then in step 212 other diagnostic tests may be applied to determine the condition of the patient.

If an ectopic event is determined in step 200, then in step 220 profiles are retrieved from memory 64 corresponding to ectopic events. In step 222 the profiles are compared to the array corresponding to the latest wavefront and if match is detected then appropriate ectopic therapy is applied in step. Otherwise other diagnostic tests for ectopic events are performed in step 226.

As discussed above, FIG. 8 shows the respective wavefronts for a sinus and an ectopic event. These events can be translated into a simple matrix using the respective time interval between the signal from each electrode and the signal from the preceding electrode. An array of this type looks like this for an intrinsic event:

INTRINSIC EVENT

| | | | | | |
|---|---|---|---|---|---|
| Electrode | 1 | 2 | 3 | 4 | 5 |
| Interval time (ms) | 0 | 20 | 20 | 10 | 15 |

The corresponding array for the ectopic wavefront of FIG. 10 looks as follows:

| | | | | | |
|---|---|---|---|---|---|
| Electrode | 4 | 3 | 5 | 2 | 1 |
| Interval time (ms) | 0 | 40 | 5 | 30 | 45 |

In these examples, the arrays consist of the electrodes listed in the order in which their respective signals are sensed and a time-dependent parameter. In the examples, given above, the time dependent parameter is the interval between the pulse on a particular electrode and the previous pulse.

The process set forth in FIGS. 16-18 is sufficient for many events and signals. However, under certain circumstances, the described process may not operate adequately without additional processing.

For example, if two arrays are determined for a patient at rest and during exercise, the interval times between these two arrays will be different. However, the percentage times will be approximately the same. (The term percentage time is defined more fully below.

Moreover, isochronal signals (signals that occur almost simultaneously on two different electrodes) may cause signals be interpreted incorrectly by the process described so far. However another analysis algorithm or process can use the conduction velocity of the propagating wavefront to provide more indicia of these latter events, as discussed below.

Wavefronts that follow the normal conduction pathway in the ventricle progress at 1.5 to 4 m/s over the conduction system, and then travel across the endocardium at the cellular conduction velocity of 30-50 cm/s. The faster conduction results in near simultaneous stimulation of large sections of the endocardium, with slower conduction stimulation propagating therefrom. Wavefronts that do not follow the normal conduction pathway travel at 30-50 cm/s across the entire heart. This differential velocity can be used as an additional indicia for ventricular events.

Atrial velocity is more difficult to determine because of the less sophisticated conduction system and the smaller size, but characteristics of the velocity can still be determined.

Again, using the example wavefronts, the intrinsic event has conduction intervals ranging from 10 to 20 ms between electrodes, while the PVC has inter-electrode intervals ranging from five to 45 ms. This indicates that there is slower conduction with the second event, typical of an ectopic event. Additionally, the entire event durations, 65 ms vs. 120 ms' Indicates a significant difference between wavefronts.

The event duration algorithm can be fooled during exercise, or other physiologic events that affect the conduction velocities in the heart. Normal sinus rhythm during exercise will present a shorter duration event than at rest, and basing a normalcy decision solely on duration would declare exercise events as ectopic. Possible algorithm improvements or additions that can address this case are discussed below. Isochrones can be identified by sorting the wavefront by absolute time. Isochrones will appear next to each other. An interval, isoX, must be defined that is based on conduction velocities and electrode spacing, and represents the confidence interval for any time event. Events within isoX are statistically simultaneous. When isochrones are identified as the first two electrodes of an event, comparisons to other wavefronts in the database must use either electrode as the initial electrode. For simplicity purposes, wavefronts stored can use the convention that with isochrones, the electrode of the smaller number will be represented first with the average time used for both. The choice for isoX is based on the electrode spacing, conduction velocity, the amount of expected variability in electrode position and conduction velocities, and the measurement resolution. Using an average conduction velocity of 30-50 cm/s, the wavefront moves 0.3-0.5 mm in 1 ms.

If electrodes are more than 5 mm apart, a time difference of 10-17 ms is expected between electrodes. With 1 ms resolution of timing for events, this interval can be easily measured with acceptable accuracy. An isoX in the range of 10% of the expected values, or 1 ms, is near the measurement resolution, so a value of isoX of 2 ms. would be appropriate. The closer electrodes are placed, the finer the required measurement resolution and the smaller isoX can be. Electrodes become very stable within the heart after several weeks, so electrode movement is also not expected. Electrode movement in the 0.5 mm range would cause changes of 1-1.5 ms. in the recorded interval for that electrode. This is actually much larger than expected, and is within the previously identified value for isoX, so no change in the preferred value for isoX needs to be made. If electrodes are placed closer, resulting in a smaller isoX, electrode movement may become an issue. Excluding physiologic events that directly affect conduction velocity, and are handled by different means, a nominal value representing the expected range of conduction velocities plus of a few percent can be selected to allow for small variations that may occur and some measurement error. If the electrodes are 5 mm apart, intervals of 10-16 ms. would be expected between electrodes, so a confidence interval of 10%, or 2 ms. could be used. Changes greater than this would indicate significant changes in conduction velocity and should be observed as such. Again, this is within the previously calculated value, so no change is required. Had there been differences in the values calculated for isoX, the larger value would have to be used, wasting the increased resolution offered by those measurements that could use a smaller isoX. The parameter isoX is thus used to handle isochronic events.

These principles are illustrated by the following examples.

### EXAMPLE NO. 1

| Wavefront 1-sinus event-patient at rest | | | | | |
|---|---|---|---|---|---|
| Electrode | 1 | 2 | 3 | 4 | 5 |
| Interval time (ms) | 0 | 20 | 20 | 10 | 15 |
| Absolute time (ms) | 0 | 20 | 40 | 50 | 65 |
| Percent time | 0 | 31 | 62 | 77 | 100 |
| Wavefront 2-sinus rhythm-patient is doing exercise | | | | | |
| Electrode | 1 | 2 | 3 | 4 | 5 |
| Interval time (ms) | 0 | 18 | 18 | 09 | 14 |
| Absolute time (ms) | 0 | 18 | 36 | 45 | 59 |
| Percent time | 0 | 31 | 61 | 76 | 100 |
| Wavefront 3-Ectopic event- patient at rest | | | | | |
| Electrode | 4 | 3 | 5 | 2 | 1 |
| Interval time (ms) | 0 | 40 | 5 | 30 | 45 |
| Absolute time (ms) | 0 | 40 | 45 | 75 | 120 |
| Percent time | 0 | 33 | 38 | 63 | 100 |
| Wavefront 4-Ectopic event- patient exercising | | | | | |
| Electrode | 4 | 3 | 5 | 2 | 1 |
| Interval time (ms) | 0 | 36 | 5 | 27 | 40 |
| Absolute time (ms) | 0 | 36 | 41 | 68 | 108 |
| Percent time | 0 | 33 | 38 | 63 | 100 |

For this example the array stored for each event consists of the list of electrodes arranged in the order in which the respective signals are sensed, the three other entries for each electrode: the interval time (between the signal on the respective electrode and the signal on the previous electrode on the list), the absolute time (between the signal on the respective electrode and the first signal) and the percent time (the absolute time expressed in percentages). A comparison between the arrays of wavefronts 1-4 illustrates that while the interval times and the absolute times for each type of event are different, the percentage times almost exactly the same. Small errors in each interval can accumulate using absolute time. Using intervals rather than absolute time eliminates this error buildup and can be used to improve accuracy.

However, as this example illustrates, the percentage time is the best parameter to include in the array to detect arrhythmia since it is independent of whether the patient is at rest, or exercising.

### EXAMPLE 2

| Wavefront 5-Sinus Event-at rest | | | | | |
|---|---|---|---|---|---|
| Electrode | 1 | 2 | 3 | 4 | 5 |
| Absolute time 0 | 20 | 40 | 50 | 65 | |
| Percent time | 0 | 31 | 62 | 77 | 100 |
| Wavefront 6-Ectopic event (PVC) | | | | | |
| Electrode | 4 | 3 | 5 | 2 | 1 |
| Absolute time 0 | 40 | 45 | 75 | 120 | |
| Wavefront 7-Ectopic event (PVC) | | | | | |
| Electrode | 4 | 3 | 5 | 2 | 1 |
| Absolute time 0 | 38 | 43 | 73 | 123 | |
| Wavefront 8-Ectopic event (PVC) | | | | | |
| Electrode | 4 | 5 | 3 | 2 | 1 |
| Absolute time 0 | 42 | 44 | 70 | 125 | |

| Wavefront 9-Sinus event-during exercise | | | | | |
|---|---|---|---|---|---|
| Electrode | 1 | 2 | 3 | 4 | 5 |
| Absolute time 0 | 18 | 36 | 45 | 59 | |
| Percent time | 0 | 31 | 61 | 76 | 100 |

The arrays can be ordered with the electrodes having the same, or standard position, as follows:

| | | | | | |
|---|---|---|---|---|---|
| Electrodes | 1 | 2 | 3 | 4 | 5 |
| Wavefront 5 | 0 | 20 | 40 | 50 | 65 |
| Wavefront 6 | 120 | 75 | 40 | 0 | 45 |
| Wavefront 7 | 123 | 73 | 38 | 0 | 43 |
| Wavefront 8 | 125 | 70 | 44 | 0 | 42 |
| Wavefront 9 | 0 | 18 | 36 | 45 | 59 |

In this situation, an isoX of 10 ms is used and, in accordance with the discussion above, differences of less than 10 ms will be assumed to be isochronal and be considered simultaneous.

Obviously numerous modifications may be made in the invention, without departing from its scope as defined in the appended claims.

I claim:

1. An implantable cardiac device for applying therapy to the heart of a patient, comprising:
a lead having a proximal end and a distal end with a plurality of electrodes, said lead being adapted to be implanted with said plurality of electrodes being positioned at various locations within the heart;
a sense detection circuit adapted to detect a plurality of sense signals from the respective electrodes; and
a control circuit adapted to analyze a timing of said sense signals and to generate an indication signal indicative of a condition of the heart based on said timing.

2. The device of claim 1 wherein said plurality of sense signals define a wavefront and wherein said control circuit is adapted to analyze said wavefront.

3. The device of claim 1 wherein said control circuit generates said indication signal to indicate one of a sinus and an ectopic cardiac event based on said wavefront.

4. The device of claim 3 further comprising a memory storing standard wavefront profiles, wherein said control circuit is adapted to generate said indication by comparing said wavefront to said standard profiles.

5. An implantable cardiac device comprising:
a plurality of at least three electrodes adapted to be disposed at different locations within the heart;
a sensor circuit adapted to sense intrinsic activity through said electrodes and to generate corresponding sense signals; and
a control circuit adapted to receive said sense signals and to detect from said sense signals a wavefront propagating through the heart tissues.

6. The device of claim 5 wherein said control circuit is adapted to determine the time at which each sense signal is sensed, said times being used to generate an array.

7. The device of claim 6 wherein said control circuit is adapted to detect a first electrode corresponding to the electrode at which the first signal of a specific wavefront is detected.

8. The device of claim 7 wherein said control circuit is adapted to differentiate between sinus and ectopic signals based on the location of said first electrode.

9. In an implantable cardiac device including a plurality of at least three electrodes constructed and arranged to be positioned at various locations within the cardiac chambers, and a control circuit connected to said electrodes, a method of diagnosing a condition of the heart, comprising the steps of:
sensing an intrinsic event at each of a plurality of electrodes;
generating a wavefront profile corresponding to said events; and
analyzing said wavefront profile to determine a cardiac condition.

10. The method of claim 9 wherein said step of generating said wavefront profile includes sensing a peak of each intrinsic event.

11. The method of claim 10 wherein said step of generating said wavefront profile includes sensing a time duration associated with said peak.

12. The method of claim 11 wherein said step of generating said wavefront profile includes assigning a position in the wavefront profile for each intrinsic event based on said time duration.

13. The method of claim 11 wherein said step of generating said wavefront profile includes assigning a position in the wavefront profile for each intrinsic event based on a ratio of said time duration to the interval length of a cardiac cycle.

14. The method of claim 9 wherein said step of analyzing includes determining a first electrode at which a cardiac activity is sensed.

15. The method of claim 14 wherein said patient condition is diagnosed as one of an ectopic and sinus condition based on said first electrode.
